(19) United States
(12) Patent Application Publication
(10) Pub. No.: US 2012/0220540 A1
(43) Pub. Date: Aug. 30, 2012

(54) SYNBODIES TO AKT1

(75) Inventors: Stephen A. Johnston, Tempe, AZ (US); Christopher Diehnelt, Tempe, AZ (US); Nidhi Gupta, Phoenix, AZ (US); Paul Belcher, Boston, MA (US)

(73) Assignee: ARIZONA BOARD OF REGENTS FOR AND ON BEHALF OF ARIZONA STATE UNIVERSITY, SCOTTSDALE, AZ (US)

(21) Appl. No.: 13/410,162

(22) Filed: Mar. 1, 2012

Related U.S. Application Data
(63) Continuation-in-part of application No. PCT/US10/47824, filed on Sep. 3, 2010.
(60) Provisional application No. 61/257,408, filed on Nov. 2, 2009.
(60) Provisional application No. 61/239,990, filed on Sep. 4, 2009.

(51) Int. Cl. A61K 38/16; A61P 35/00; C07K 14/00; G01N 33/573

(52) U.S. Cl. 514/19.4; 435/7.4; 514/19.3; 514/19.5; 530/323; 530/324

(57) ABSTRACT

The present application provides synbodies against AKT1 differing in amino acid sequence, conjugation chemistry, linker/scaffold, or adjunct moiety. The synbodies are useful for diagnosis and treatment of cancer and as research reagents.

### BACKGROUND OF THE INVENTION

AKT1 is a protein kinase that phosphorylates serine or threonine residues in many proteins, mediating the effects of several growth factors, including EGF and IGF-1. AKT1 is associated with a variety of physiological responses, including insulin-stimulated protein synthesis, inhibition of apoptosis and promotion of cell survival. AKT1 has been associated with a tumorigenesis, tumor invasiveness and chemoresistance. Elevated levels have been reported in breast cancer, ovarian cancer, pancreatic cancer (Bellacosa et al., Adv Cancer Res. 2005; 94:29-86), and a prostate cancer cell line (see, e.g., Nakatani et al. J. Biol. Chem. 274, 21528-21532 (1999)). AKT1 is also activated by the BCR/ABL fusion gene in chronic myelogenous leukemia (see, e.g., Thompson and Thompson. J. Clin. Oncol. 22, 4217-26 (2004)). AKT1 activity can be abnormally activated, for example, as a result of duplication of an AKT gene, overexpression of an AKT gene or protein, or abnormal activation of an AKT signal transduction pathway.

Several antibodies targeted to other tumor antigens (e.g., Herceptin, Mylotarg, Avastin, Erbitux) have been approved for clinical use, and have achieved at least modest success in extending the life of patients suffering from various types of cancer in which the relevant target antigen is expressed.

WO08/048970 describes methods for isolating a class of molecules termed synthetic antibodies or synbodies. Synbodies contain at least two compounds, such as short peptides, joined via a linker. Although the affinity of individual compounds for a target is typically weak, the combination of compounds can bind desired target with affinities comparable to antibodies. Synbodies have advantages over antibodies resulting in part from their smaller size. These advantages may include ease of initial isolation, ease and cost of production, and improved tissue penetration.

### BRIEF SUMMARY OF THE INVENTION

The invention provides an agent comprising a first peptide having an amino acid sequence comprising AX₁KVVX₂QRX₃X₄RX₅AYX₆RYGSG (SEQ ID NO: 1), wherein X₁ is H or W, X₂ is P or Y, X₃ is Q or W, X₄ is I or M, X₅ is H, Y or F, and X₆ is N or S, and a second peptide having an amino acid sequence comprising FRGWAHIFFGPHVIYRGGSG (SEQ ID NO: 3), and a linker joining the first and second peptides, the three C-terminal amino acids and up to five other amino acids can be substituted for, inserted or deleted in the first and/or second peptides at positions not indicated by X, and provided that the first peptide does not have an amino acid sequence consisting of AHKVVPQRQIRHAYNRYGSG (SEQ ID NO: 2) or the first and second peptides are not linked via amide bonds to alpha and epsilon amino groups of a lysine linker. Optionally, the agent has an affinity for human AKT1 of at least 10⁸ M⁻¹ or at least 10⁹ M⁻¹. Optionally, the first and second peptides are linked in a MAP format. Optionally, the linker is an amino acid, peptide, polymer, a cyclic compound, or a particle. Optionally, the linker is a lysine, dilysine, lysine-cysteine, PGP, PEG, a sequential oigo-peptide carrier, a templated assembled scaffold, amino biphenyl carboxylic acid, a calyx(n)arene, triazacylophane, beta-cyclodextrine, a nanoparticle, a gold particle, or a quantum dot. Optionally, the agent comprises at least two molecules of the first and/or second peptide, wherein each peptide is linked to the linker. Optionally, the linker is polylysine.

Some agent further comprise a label, immobilizing moiety, therapeutic molecule or half-life extender. Optionally, the label, immobilizing moiety, therapeutic molecule or half-life extender is attached to the linker. Optionally, the immobilizing moiety is biotin. Optionally, the therapeutic molecule is a cytotoxic molecule. Optionally, the half-life extender is selected from PEG, phosphorylcholine and an immunoglobulin constant region. The invention further provides a method of detecting AKT1, comprising contacting a sample suspected of containing AKT1 with an agent as defined above, and measuring binding of the agent to the sample compared with a control lacking AKT1, an increase in binding relative to the control providing an indication of presence of AKT1. Optionally, the sample is from a patient having or suspected of having a cancer or elevated risk of cancer. Some methods further comprise contacting the sample with a second agent that binds a different epitope of AKT1, wherein either the first or second agent is immobilized and wherein the measuring step detects a sandwich formed between the first agent, AKT1 and the second agent.

The invention further provides a method of inhibiting growth of a cancer comprising, contacting the cancer with the agent as defined above. Optionally, the agent further comprises a therapeutic molecule linked to the linker. In some methods, cancer is present in a patient, optionally, ovarian, breast or prostate cancer.

### BRIEF DESCRIPTION OF THE DRAWINGS

FIG. 1 shows a lead synbody with 2 20-amino acid peptides attached to a lysine linker, which is in turn bonded to a second lysine and biotin.

FIG. 2A shows SPR sensorgrams of the lead synbody and immobilized AKT1. FIG. 2B shows a Western Blot of immunoprecipitation of AKT1 using the synbody. FIG. 2C shows a plot of S35-labeled AKT1 pulled-down with synbody immobilized via its biotin to a magnetic bead. FIG. 2D) shows a Western Blot of 200 ng of recombinant AKT1 pulled down in the presence of increasing concentrations of A549 cell lysate. FIG. 2E shows immunoprecipitate of purified 250 ng of AKT1, AKT2, and AKT3 from 500 mg of cell lysate.

FIG. 3 shows a synbody binding to cells expressing AKT1.

FIG. 4 shows exemplary synthetic schemes for producing synbodies.

FIG. 5 shows a lysine scaffold displaying multiple copies of one of the compounds of a synbody.

FIG. 6 shows a synbody dimerized via disulfide bonding.

FIG. 7 shows synbodies including varying spacer lengths between a lysine linker and compounds displayed from the linker.

FIG. 8 shows a synbody in which compounds are displayed from a non peptide calyx[n]rene scaffold.

FIGS. 9 and 10 show synbodies in which compounds are displayed from linear (FIG. 9) and cyclic (FIG. 10) peptide backbones.

FIG. 11 shows a synbody in which compounds are displayed from a Quantum dot.

FIGS. 12A and 12B shows synbodies conjugated by copper catalyzed alkyne azide cycloaddition to introduce various lengths of spacer between the linker and compounds.

FIG. 13 shows a synbody conjugated via thiazole chemistry.

FIG. 14 shows a synbody conjugated via thiazolidine chemistry.

### DEFINITIONS

A synbody is a synthetic entity having at least three components, two of which are compounds having affinity for the same target molecule albeit at different sites within the target molecule and the third being a linker connecting the compounds. The molecular weight of a synbody is usually 500-10,000 kDa and sometimes between about 4 and 5 kDa.

A linker indicates a moiety or group of moieties that connects two or more discrete compounds in a synbody. A linker is typically bifunctional (i.e., the linker contains a functional group at each end that is reactive with groups located on the compounds to be attached). Linkers include amino acids, polypeptides, nucleic acids, small molecules, polymers and particles. Linkers can be linear or branched. Particles serving as linkers or linkers attached to multiple copies of the compounds forming a synbody are sometimes referred to as scaffolds.

A spacer is a molecule optionally present between a linker and a compound attached to the linker. A spacer can be, for example, one or more amino acids or a small organic structure conjugating the linker to a compound.

In some synbodies, the demarcation of compounds, linker and spacer(s) if present is readily apparent, because each has a contiguous or regularly repeating structure distinct from another, or because of conjugation chemistries indicating the points of demarcation. However, a precise understanding of demarcation between these components is not usually necessary for use.

An isolated peptide or other moiety means that the moiety if found in nature is separated at least in part from the molecules with which it is naturally associated including flanking sequences if the peptide is part of a longer protein. If the peptide or moiety is synthetic, isolated means separated at least in part from chemicals used in its production. An isolated peptide does not exclude the presence of heterologous components, such as a linker, second peptide or pharmaceutical excipients not naturally associated with the peptide or used in its synthesis. An isolated moiety can also be pure (e.g., at least 50, 75, 90 or 99%% w/w pure) of contaminants

Unnatural amino acids are amino acids other than the twenty naturally occurring amino acids that are the building blocks for all proteins, but are nonetheless capable of being biologically or chemically engineered such that they are incorporated into proteins. Unnatural amino acids include D-amino acids, β amino acids, and various other “designer” amino acids (e.g., β-methyl amino acids, Cα-methyl amino acids, and Nα-methyl amino acids. Synthetic amino acids include ornithine for lysine, and norleucine for leucine or isoleucine. Hundreds of different amino acid analogs are commercially available from e.g., PepTech Corp., MA. In general, unnatural amino acids have the same basic chemical structure as a naturally occurring amino acid, i.e., an a carbon that is bound to a hydrogen, a carboxyl group, an amino group, and an R group. Methods of making and introducing a non-naturally-occurring amino acid into a protein are known. See, e.g., U.S. Pat. Nos. 7,083,970; and 7,524,647. Some unnatural amino acids

Derivatives should have a stabilized electronic configuration and molecular conformation that allows key functional groups to be presented to the target binding sites in substantially the same way as the lead multimer. Identification of derivatives can be performed through use of techniques known in the area of drug design. Such techniques include self-consistent field (SCF) analysis, configuration interaction (CI) analysis, and normal mode dynamics analysis. Computer programs for implementing these techniques are readily available. See Rein et al., Computer-Assisted Modeling of Receptor-Ligand Interactions (Alan Liss, N.Y., 1989). Derivatives may have higher binding affinity, smaller size, and/or improved stability relative to a lead multimer. Modifications can include N terminus modification, C terminus modification, peptide bond modification, including, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference.

Specific binding refers to the binding of a compound to a target (e.g., a component of a sample) that is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however imply that a compound binds one and only one target. Thus, a compound can and often does show specific binding of different strengths to several different targets and only nonspecific binding to other targets. Preferably, different degrees of specific binding can be distinguished from one another as can specific binding from nonspecific binding. The peptides and synbodies of the invention show specific binding to human AKT1. Specific binding of synbodies of the invention usually involves an association constant of 10⁷, 10⁸ or 10⁹ M⁻¹ or higher.

### DETAILED DESCRIPTION OF THE INVENTION

I. General

WO08/048970 and PCT/US2009/041570 provides a first generation synbody binding to AKT1. The present application provides variants of this synbody differing in e.g., amino acid sequence, conjugation chemistry, linker/scaffold, or adjunct moiety.

II. AKT1

AKT1 (e.g., UniProtKB/Swiss-Prot P31749 (AKT1_HUMAN)) (SEQ ID NO: 4) is a well known serine-threonine kinase associated (usually by elevated expression) with many forms of cancer KT1 is part of a family of genes that also includes AKT2 (UniProtKB/Swiss-Prot P31751) (SEQ ID NO: 5) and AKT3 (UniProtKB/Swiss-Prot Q9Y243) (SEQ ID NO: 6).

III. Synbodies

A. Basic Structure

The first generation or lead synbody has two twenty-amino acid peptides Akt26 (AHKVVPQRQIRHAYNRYGSG) (SEQ ID NO: 2) and Akt23 (FRGWAHIFFGPHVIYRGGSG) (SEQ ID NO: 3) attached to the Nα and Nε-positions respectively of a lysine.

B. Amino Acid Variants

Some preferred amino acid variants of the lead peptides are shown in Table 1 below. These substitution variants all occur in peptide AHKVVPQRQIRHAYNRYGSG (SEQ ID NO: 2). The individual variations can be combined in different permutations represented by the formula AX₁KVVX₂QRX₃X₄RX₅AYX₆RYGSG (SEQ ID NO: 1), wherein X1 is H or W, X2 is P or Y, X3 is Q or W, X4 is I or M, X5 is H, Y or F, and X6 is N or S. The three C-terminal amino acids represent a tag to facilitate attachment of the peptide and any or all can be replaced or deleted without substantially affecting binding affinity. Up to 1, 2, 3, 4, or 5 other amino acids can be substituted for, inserted or deleted in the peptide. The substitutions, deletions or insertions can be performed at positions indicated by X or at positions not indicated X or at a combination of positions indicated by X and not indicated by X. Some peptides contain entirely natural amino acids and peptide linkages. However, substitutions can also be performed with unnatural amino acids. Alternatively, natural amino acids can be derivatized post incorporation into a peptide, for example, at the free terminus not linked to a linker.

TABLE 1

| | |
|---|---|
| AHKVVPQRQMRHAYSRYGSGKCFRGWAHIFFGPHVIYRGGSG | (SEQ ID NO: 7) |
| AHKVVYQRQIRFAYNRYGSGKCFRGWAHIFFGPHVIYRGGSG | (SEQ ID NO: 8) |
| FRGWAHIFFGPHVIYRGGSGKCAHKVVYQRQIRFAYNRYGSG | (SEQ ID NO: 9) |
| AWKVVPQRQIRHAYNRYGSGKCFRGWAHIFFGPHVIYRGGSG | (SEQ ID NO: 10) |
| AHKVVPQRWIRHAYNRYGSGKCFRGWAHIFFGPHVIYRGGSG | (SEQ ID NO: 11) |
| AHKVVPQRWIRHAYNRYGSGKCFRGWAHIFFGPHVIYRGGSG | (SEQ ID NO: 12) |
| AWKVVPQRWIRYAYNRYGSGKCFRGWAHIFFGPHVIYRGGSG | (SEQ ID NO: 13) |

Additional substitutions, deletions or insertions can be made in addition to those specifically indicated above in either or both of the peptides. For example, the tri-peptide GSG occurring at the C-terminus of each peptide is amenable to substitution, for example, with KSG, GSC or KSC. Usually a total of five or fewer changes (substitution, deletion or insertion) is sufficient to optimize binding of a peptide. Some peptides have no more than 4, 3, 2 or 1 or zero changes other than those in the formula given above. Flanking sequences, for example, peptide tags or spacers can also be attached to the peptides, without significantly affecting binding to AKT1.

Several approaches for making and testing variants of synbodies have been described (see WO08/048970 and PCT/US2009/041570). Peptide components can be optimized individually or together as synbody. The optimization can be performed by making a population of variants of a peptide, and screening or selecting the variants for binding to the target. In some methods, known as linear optimization, a single position in each peptide is varied at a time in a first round and the variants showing greatest improvement in binding are combined in subsequent rounds. Another approach is alanine scanning mutagenesis. Another approach is to delete amino acids from the ends or internally to identify amino acids that contribute little if anything to binding. Variants can be screened by surface plasmon resonance or display techniques among others. That is, each variant tested differs from an initial peptide at a single position, although the position may vary in different peptides, such that most or all positions in an initial peptide are varied.

The synbodies of the present invention can further comprise an adjunct moiety. Exemplary adjunct moieties include a label, immobilizing moiety, therapeutic molecule or half-life extender. The adjunct moieties are usually attached to the linker, although they can be attached to the first peptide or the second peptide as well or instead.

Synbodies of the present invention can have multiple copies of a first peptide and multiple copies of a second peptide. For example, a synbody can have 1-3 copies of a first peptide and 1-3 copies of a second peptide. Some synbodies have one copy of a first peptide and one to three copies of a second peptide. Other synbodies have one to three copies of a first peptide and one copy of a second peptide.

The synbodies of the present invention can further comprise a spacer between one or both of the peptides and the linker. Exemplary spacers include octanoic acid (Oct), hexanoic acid, polyethylene glycol, poly-(proline-glycine-proline), or β-amino hexanoic acid. In some embodiments of the invention, the spacer is octanoic acid (Oct).

C. Conjugation Chemistry Variants

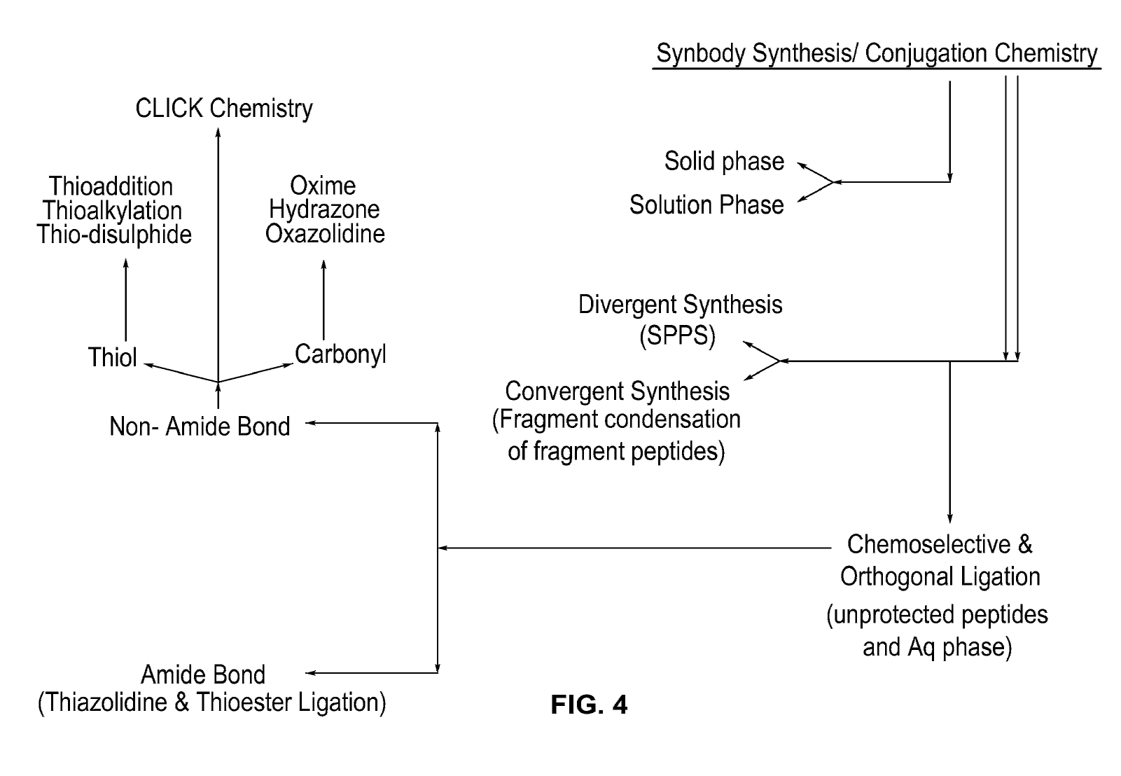

FIG. 4 is a schematic showing some of the available types of synthesis scheme and conjugation chemistry for synthesizing peptides and linking the peptides to a linker or scaffold.

Synthesis of the synbodies described herein may employ solid phase synthetic methods, solution phase synthetic methods, and/or combinations of both solid phase and solution phase synthetic methods. In some embodiments of solid phase and/or solution phase synthetic strategies, a divergent synthesis method is employed. The term “divergent synthesis method” refers to a method in which libraries of complex compounds (e.g. a synbody) are generated by reaction of a core molecule with a set of reactants to provide a plurality of first generation compounds. Each first generation compound is then further reacted with a set of reactants to provide a second generation of compounds. The process can be reiterated to provide subsequent generations of compounds resulting in a set or plurality of complex compounds. This methodology quickly diverges to large numbers of new complex compounds. The term “core molecule” in this context refers to a chemical species which is common to the first, second and subsequent generations of compounds as well as the resulting complex compounds. For example, a core molecule may be a chemical scaffold that includes orthogonally protect amino acid side chains.

In contrast to divergent synthesis methods, in some embodiments of solid phase and/or solution phase synthetic strategies, a convergent synthesis method is employed. The term “convergent synthesis” may refer to the process of linking together chemical moieties (also referred to herein as “chemical elements”) to form the complex compound (e.g. synbody). In some embodiments, the various chemical elements are linked to a core molecule. Convergent synthetic strategies are commonly employed in the synthesis of dendrimers. See for example Pittelkow M & Christensen B, Organic Letters, 2005, 7:1295-1298, which is incorporated by reference herein and for all purposes. A chemical element may refer to portions or fragments of proteins, nucleic acids, peptides and small molecules which become incorporated during synthesis of the synbodies described herein. In some embodiments, the chemical element is a peptide, amino acid, amino acid side chain, or fragment thereof. In some embodiments, the chemical element is solid-phase-bound during solid phase synthesis. Further exemplary chemical elements include affinity chemical elements as described herein. In some embodiments, the chemical element is a protected or unprotected peptide, amino acid, amino acid side chain or fragment thereof. In some embodiments, one or more reactive groups in the chemical element is protected from synthetic reactions (e.g. orthogonally protected).

A variety of methods for ligation of chemical elements are available including chemoselective ligation and/or orthogonal ligation. The term “chemoselective ligation” includes the selective covalent coupling of mutually and uniquely reactive functional groups under mild, typically aqueous, conditions. See for example Lemieux G & Bertozzi C, Trends in Biotechnology, 1998, 16:506-513, which is incorporated by reference herein and for all purposes. The term “orthogonal ligation” may refer to an amino terminal specific method for coupling chemically unprotected amino acids. Some peptide ligation methods may not require coupling reagents or protection schemes but are achieved through a variable chemoselective capture step and then an invariable intramolecular acyl transfer reaction. See for example Tam J & Eom K, Biopolymers, 2001, 60:194-205.

Bonds formed in chemoselective ligation or orthogonal ligation may be classified generally as amide bond ligation or non-amide bond ligation. In this context, a variety of methods are available. For example, the “thiazolodine method” involves reaction of a peptide aldehyde with a cysteinyl peptide to afford the thiazolidine linked compound. As shown in Scheme 1 below, a C-terminal 1,2-propanediol can be oxidized to the aldehyde. Subsequent reaction with an N-cysteinyl peptide affords the thiazolidine linked ligation compound. Alternatively, as shown in Scheme 2 below, oxidation of an N-terminal serinyl peptide can afford the N-terminal peptide aldehyde, which may then be reacted with an N-terminal cysteinyl peptide to form the thiazolidine linked ligation compound.

In some embodiments, a first peptide element having a C-terminal aldehyde is ligated to a second peptide element having an N-terminal cysteinyl residue, resulting in a thiazolidine containing bridge (also referred to herein as a thiazolidinyl linker) between the first and second peptide elements. In some embodiments, a first peptide having an N-terminal seryl residue is oxidized and subsequently reacted with a second peptide element having an N-terminal cysteinyl residue to form the bridged first and second peptide element compound. In some embodiments, compounds resulting from bond formation via the thiazolidine method become elements for subsequent ligation to form a synbody or portion thereof.

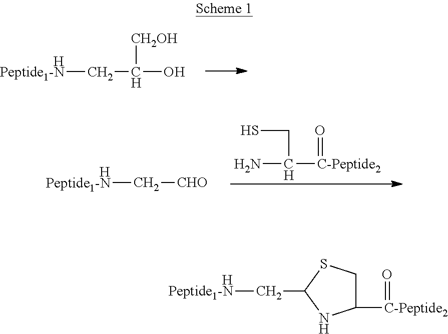

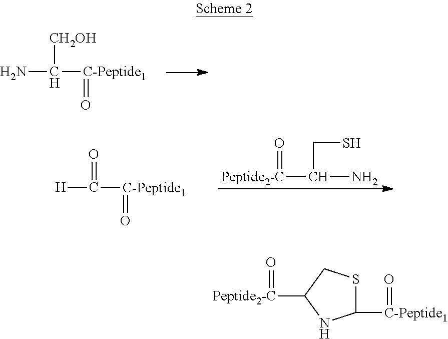

Additional strategies are available for the formation of bonds during the ligation of elements. For example, in “thioester ligation,” a first peptide containing a C-terminal thioester reacts with a second peptide containing an N-terminal cysteine, usually in the presence of an added thiol catalyst. In a reversible first step, a transthioesterification occurs to yield a thioester-linked intermediate, which intermediate rearranges irreversibly under the usual reaction conditions to form a native amide bond at the ligation site. See for example Dawson P E et al., 1994. Science 266:776-779. In some embodiments, cysteine analogs and/or homologs are used in place of cysteine to afford post-ligation synthetic opportunities including alkylation (e.g., methylation of homocysteine to form methionine), and desulfurization conversion of cysteine to alanine).

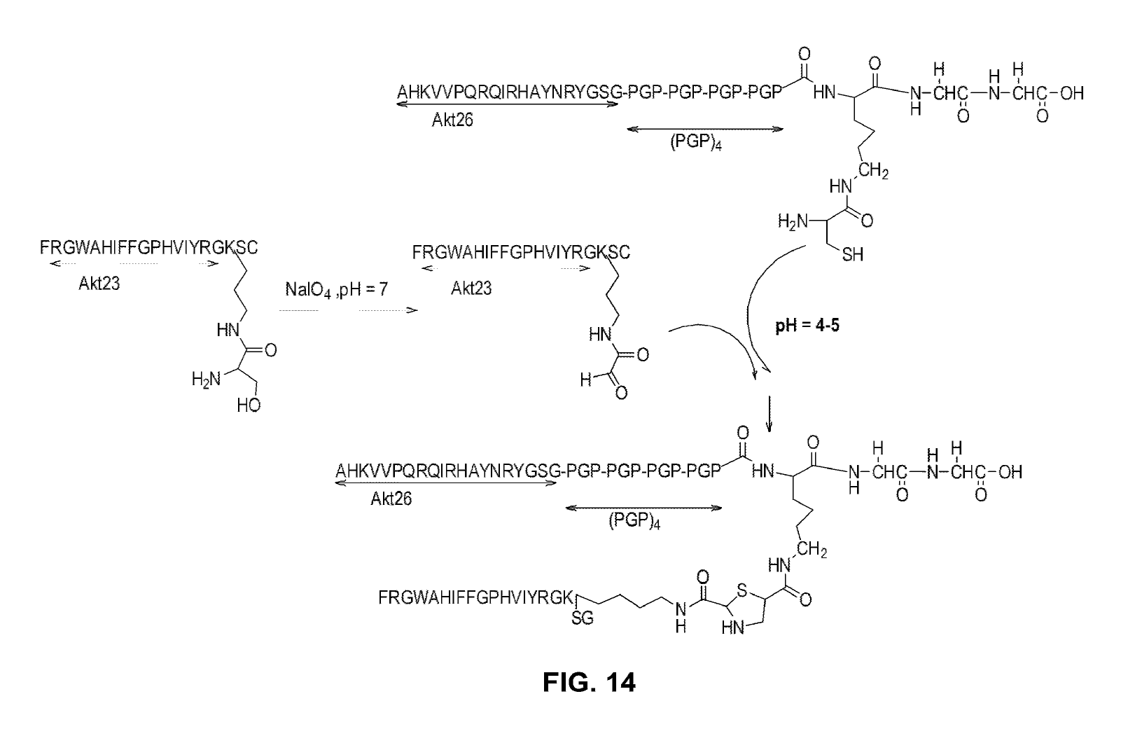

Thiazolidine chemistries may also be optimized for synthesis of certain synbodies described herein (see FIG. 14).

A variety of additional methods are available for formation of non-amide bonds in the ligation of chemical elements. Such methods include reactions employing thiol chemistry, including thioaddition, thioalkylation and thiodisulphide formation.

Additional methods of non-amide bond formation are available which exploit carbonyl chemistry. For example, iterative O-amination followed by oxime bond formation can provide a variety of polyaryl oxime species, an exemplary reaction of which is shown in Scheme 3. In Scheme 3, elongation of the growing polyaryl oxime can proceed when R′ is, for example, hydroxy. See Renaudet O & Reymond R-L, Organic Letters, 2003, 5:4693-4696. In scheme 3, R and R′ are optionally different substituents. Oxime chemistries were optimized for synthesis of certain synbodies described herein.

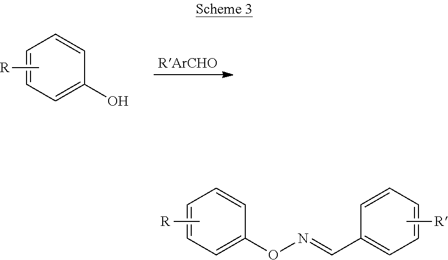

Additional methods of non-amide bond formation include, for example, use of conventional hydrazone and oxazolidine reaction chemistry.

The use of the so-called “click” chemistry provides a wealth of synthetic opportunities. For example, copper catalyzed alkyne azide cycloaddition (CuAAC) conjugation provides a means to control spacing of compounds from a linker. As shown in Scheme 4, reaction of an azide with an alkyne in the presence of Cu(I) results in thiazole bridging.

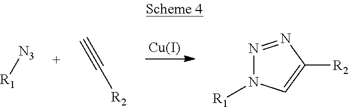

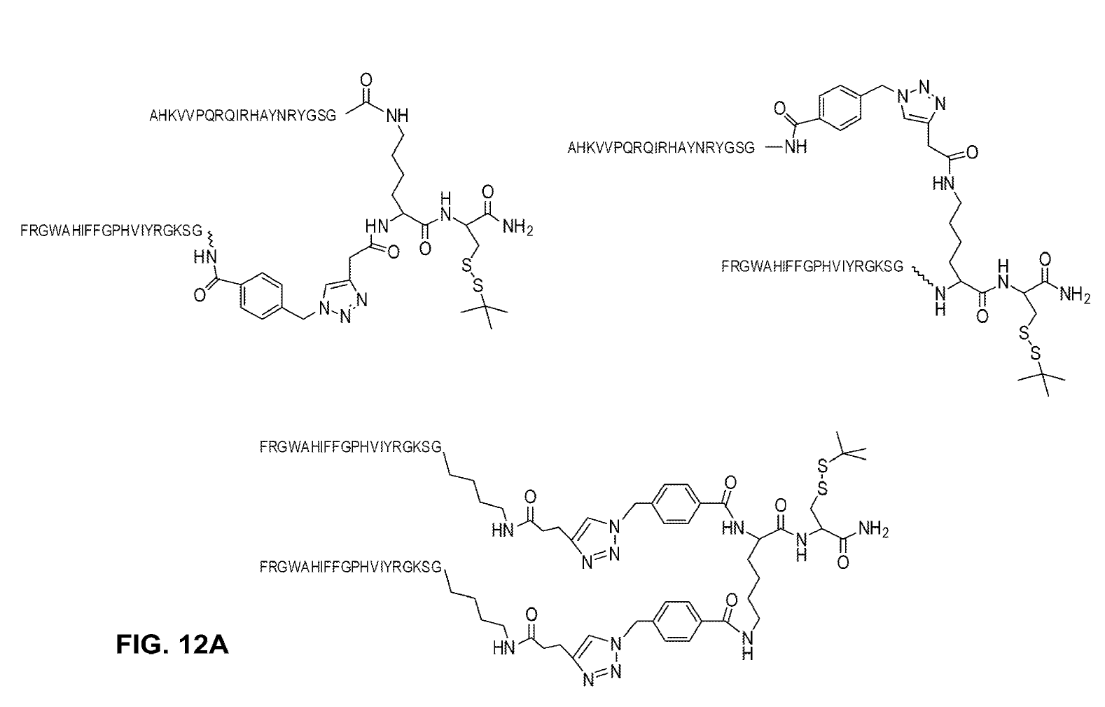

Use of CuAAC allows two orthogonal building blocks to be joined to a linker under extremely mild reaction conditions. For example, both amino groups of a lysine linker can be modified using different azido groups (e.g. 4-azidomethylbenzoic acid, 5-pentanoic acid, Fmoc-p-Phe-OH) and reacted with an alkyne group (e.g., Fmoc-Pra-OH, 4-pentynoic acid, Propiolic acid) introduced onto portions of the synbody or vice versa (see FIGS. 12A and 12B). FIG. 12A and FIG. 12B include sequences FRGWAHIFFG PHVIYRGGSG KSG (SEQ. I.D. NO: 51), FRGWAHIFFG PHVIYRGKSG (SEQ. I.D. NO: 52), AHKVVPQRQI RHAYNRYGSG KSG (SEQ. I.D. NO: 53), FRGWAHIFFG PHVIYRGGSG YSG (SEQ. I.D. NO: 54), AHKVVPQRQI RHAYNRYGSG PGPPGPPGPP GP (SEQ. I.D. NO: 55), and FRGWAHIFFG PHVIYRGKSC (SEQ. I.D. NO: 56).

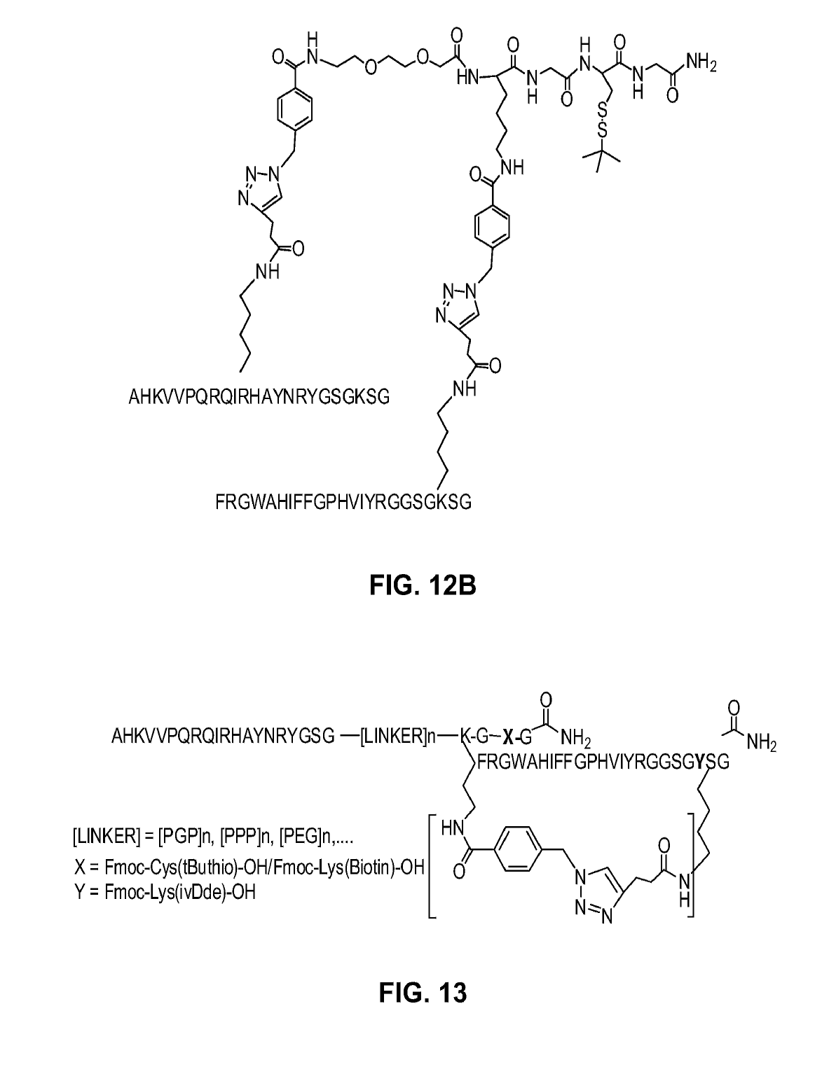

Libraries of ligands tethered via amide as well as triazole may be prepared by incorporating varying lengths of variety of linker (see FIG. 13).

Spacers between one or both of the synbody compounds and the linkers can be incorporated using strategies described herein and/or known in the art. Variation in the length of spacer provides a facile method to reach the key regions of AKT1 involved in molecular recognition. The spacers can be formed, for example, from different number and combinations of a variety of monomeric units including β-alanine and aminohexanoic acid units. In some embodiments, suitably protected monomeric or polymeric β-alanine or aminoalkyl acid (for example, aminohexanoic acid and the like) reagents are ligated via standard solid phase peptide synthesis (SPPS) methodologies, including for example Boc and Fmoc chemistries. In some embodiments, ligation of such spacer occurs in the solution phase. In some embodiments, a polymeric β-alanine- or aminoalkyl acid-containing spacer is synthesized prior to incorporation into a compound described herein.

Additional chemical strategies for the incorporation of spacers into compounds described herein include, the use of peptoids (Simon R et al., Proc. Natl. Acad. Sci. USA, 1992, 89:9367), other amino acids not containing an alpha-amino functionality (Seebach D et al., J. Chem. Soc., Chem. Commun., 1997, 21:2015), vinylogous peptides (Hagihara M et al., J. Am. Chem. Soc., 1992, 114:6568), vinylogous sulfonyl peptides (Gennari C et al., Angew. Chem. Int. Ed., 1994, 33:2067), permethylated polypeptides (Ostresh J et al., Proc. Natl. Acad. Sci. USA, 1994, 91:11138), β-sulfonyl polypeptides (Moore W et al., J. Org. Chem., 1995, 60:5157), oligoureas (Burgess K et al., Angew. Chem. Int. Ed. Engl. 1995, 34:907), oligocarbamates (Cho C et al., Science, 1993, 261:1303), oligosulfones (Moran E et al., Biopolymers, 1995, 35:213), azapeptides (Gante J, Chem. Ber. 1965, 98:3340), azatides (Han H & Janda K, J. Am. Chem. Soc., 1996, 118:2539), hydrazinoaza polymers (Cheguillaume A et al, J. Org. Chem., 1999, 64:2924) and a/b aminooxy peptoids (Shinb I & Park K, Org. Lett. 2002, 4:869).

D. Linker Variants

A variety of linkers in varying lengths ranging from rigid to flexible are available to connect peptides. For example, polyproline is a rigid linker, polyethylene glycol is a flexible linker, and Pro-Gly-Pro is of intermediate flexibility. Exemplary peptide-based linkers including multiple antigenic peptides linkers (e.g., branched lysine), [Pro-Gly-Pro]n (PGP), [Pro-Pro-Pro]n (Poly Proline), Poly(ethylene)glycol (PEG), Sequential Oligo-peptide Carriers (SOC-I & II), and template assembled scaffold, such as Cyclic PGP. Exemplary non-peptide based scaffolds include amino biphenyl carboxylic acid (ABC), triazacyclophane (TAC), Calix[n]arenes n=4, 6, β-Cyclodextrin and nano-particles, such as Q dots, and gold particles).

Some linkers allow attachment of multiple copies of one or both peptides. These linkers are known as multivalent linkers or scaffolds. Such scaffolds are sometimes better in mimicking and addition of constraint present in natural molecules. Use of a cyclic linker can improve target selectivity favoring the binding to one protein over the other that shares a high degree of homology or ligand reorganization properties. Cyclic linkers also confer increased resistance to protease.

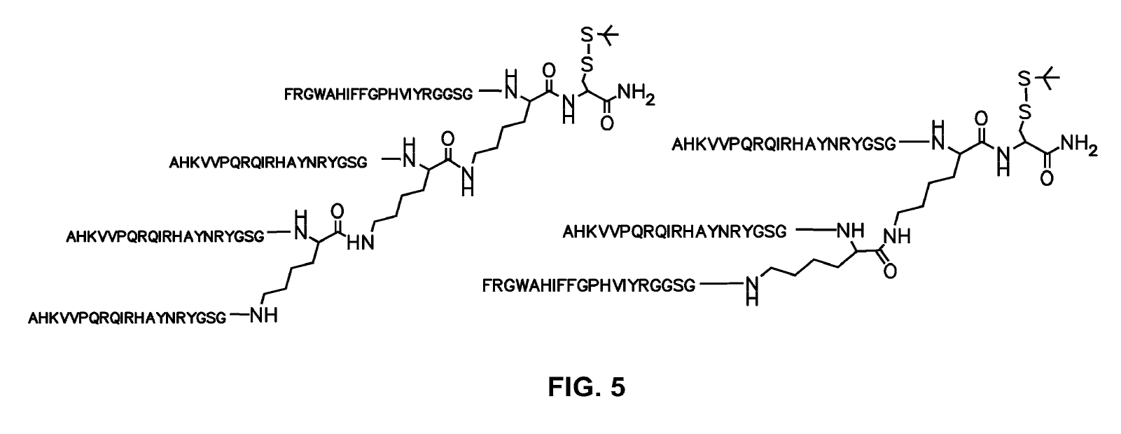

Exemplary linkers which allow attachment of one or more copies of one or both peptide elements of the synbodies described herein are illustrated in FIG. 5. In FIG. 5A, a peptide with the C-terminal sequence Lys-Cys(S-tBu)-amide is elaborated at the Nε position of the penultimate lysine with two additional lysine residues joined via Cα-Nε linkage. In some embodiments, synthesis of this species can be achieved with SPPS strategies by appropriate choice of orthogonal protection schemes as known in the art. In some embodiments, synthesis of this species can be achieved with chemical ligation methods described herein. In FIG. 5B, a single side-chain lysine is joined to the Nε position of the penultimate lysine, thus providing two additional growth points for SPPS at the indicated Nα and Nε positions. The terms “side-chain lysine,” “side-chain residue” and the like in the context of linkers refer to residues (e.g., lysine) bound at the side chain of another peptide. In some embodiments, side-chain residues are formed using standard SPPS strategies, incorporating for example Boc, Fmoc and the like. In some embodiments, side-chain residues are formed using other chemistries described herein and/or known in the art.

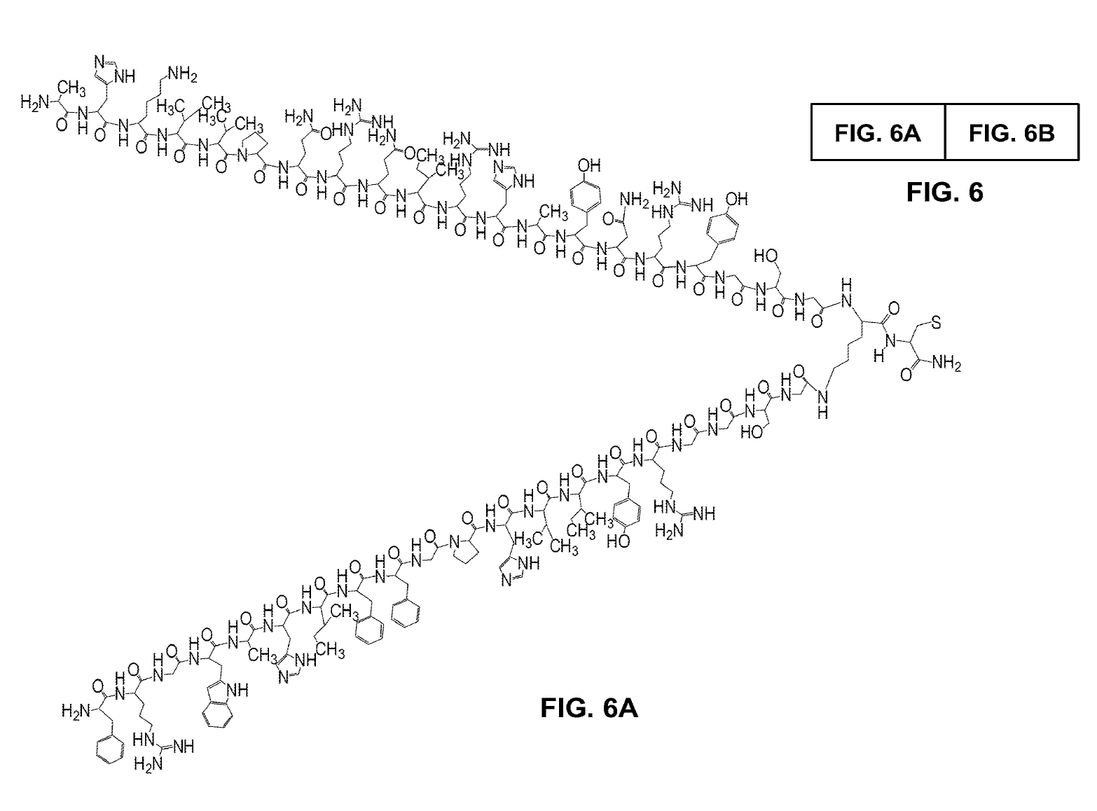

Higher order synbodies are available through a variety of methods known in the art for the condensation of synbodies. The term “higher order synbody” refers to covalently bonded assemblages of two or more synbodies as described herein. For example, FIG. 6 illustrates a higher order synbody resulting from the formation of a disulfide bond between the Sγ atoms of two synbodies. In this case, the synbodies were separately formed, one affinity element having a C-terminal Lys-Cys(S-tBu)-amide, and the other affinity element elaborated from Nε of the penultimate lysine. Synthesis of this higher-order synbody was achieved by solution phase deprotection of the C-terminal cysteine followed by disulfide bond formation, as known in the art.

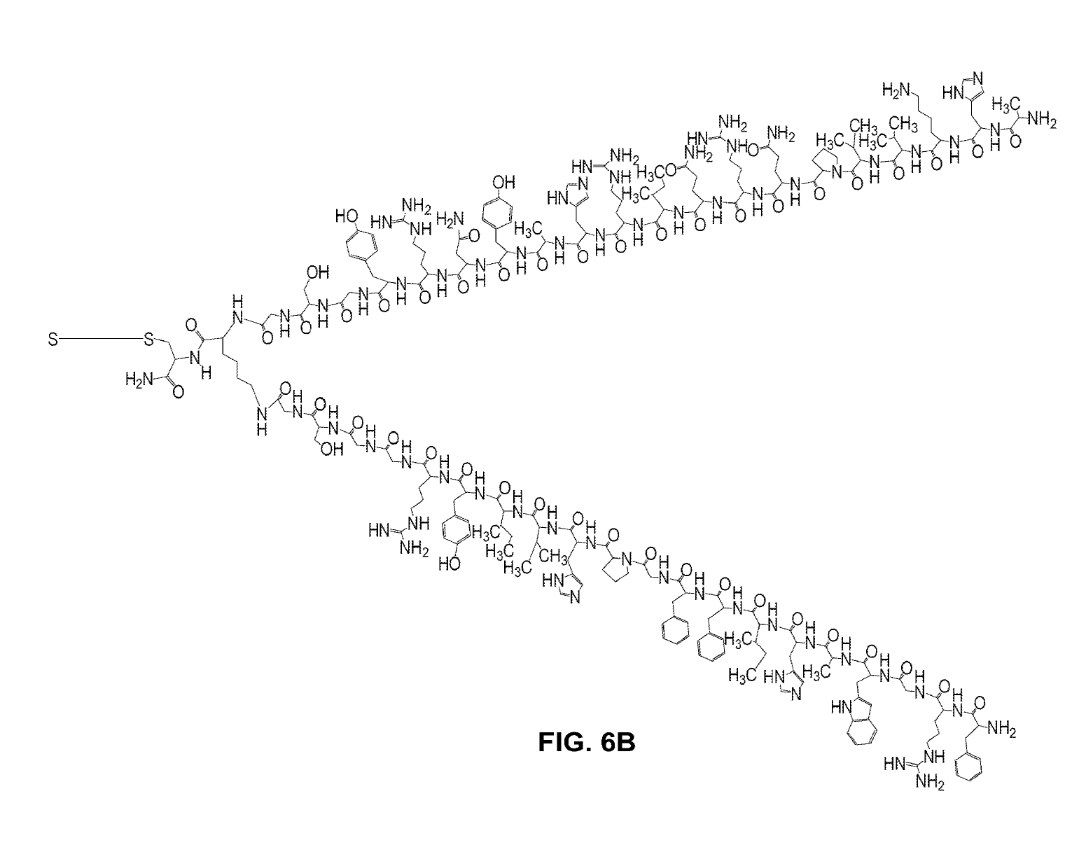

The synbodies described in FIG. 7 illustrate the considerable freedom afforded by the synthetic strategies described herein. For example, in the upper molecule of FIG. 7 a resin bound protected lys-cys dipeptide may serve to anchor subsequent synthetic steps. The lysine is typically orthogonally protected (e.g., Fmoc-Lys(ivDde)-OH, and the like) allowing for standard SPPS orthogonal protection strategies to be employed. The side chain of cysteine is typically protected with a thiol protecting agent (e.g., thio-t-butyl group and the like). A carboxy-polyethylene glycol-amine (carboxyl-PEG-amine) can be ligated to the Nα-lysine to form a growing PEG arm with a free end having a potentially reactive terminal nitrogen. A variety of protected carboxy-PEG-amine reagents are available commercially for use in SPPS, as known in the art. In some embodiments, multiple rounds of ligation with carboxy-PEG-amine are employed, for example, 1, 2, 3, 4, 5 or more. The free end of the PEG arm can be further elaborated as described herein. For example, in the upper molecule of FIG. 7, a triazole aryl containing spacer is terminated by an affinity element. Further to the upper molecule of FIG. 7, the Nε of the anchor dipeptide lysine can be ligated with orthogonally protected lysine, forming a first side chain lysine thereby providing two additional peptide growth points at the Nα and Nε nitrogens of the newly added lysine. The term “growth point” in the context of SPPS refers to a free amine capable for coupling to form an amide bond. Standard differential deprotection at these growth points and subsequent SPPS can afford the upper molecule of FIG. 7. Variations on the synthetic strategies employed for the upper molecule of FIG. 7 are available. For example, in the lower molecule of FIG. 7, Nα of the dipeptide anchoring lysine can serve as the growth point for an affinity element, and the Nε of this anchoring lysine can be coupled with an orthogonally protected first side chain lysine to provide two additional growth points. The newly available growth points can be ligated with spacers and ultimately block condensed with affinity elements to provide the lower compound of FIG. 7.

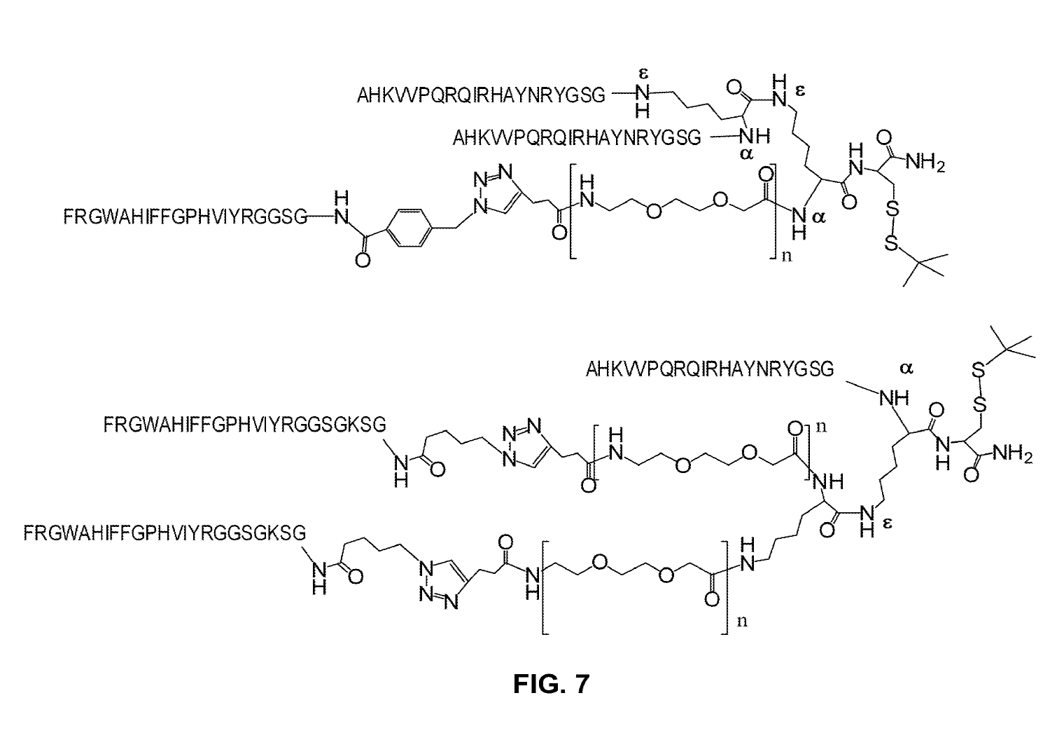

Exemplary scaffolds functioning as linkers for the affinity elements of the synbodies described herein include macrocyclic structures known in the art. For example, FIG. 8 illustrates a higher-order synbody built on a calix[4]arene scaffold. Calix[n]arenes, wherein “n” represents the number of aryl monomers forming the macrocycle, are macrocyclic polymers based on hydroxyalkylation of a suitably substituted phenol and an aldehyde. Thus, a protected reaction site at the para-position of the phenol monomers of the calix[4]arene of FIG. 8 can unmask after formation of the calix[4]arene. In some embodiments, affinity elements are ligated at such unprotected sites using chemical strategies described herein. In some embodiments, the affinity elements are synthesized in the solution phase.

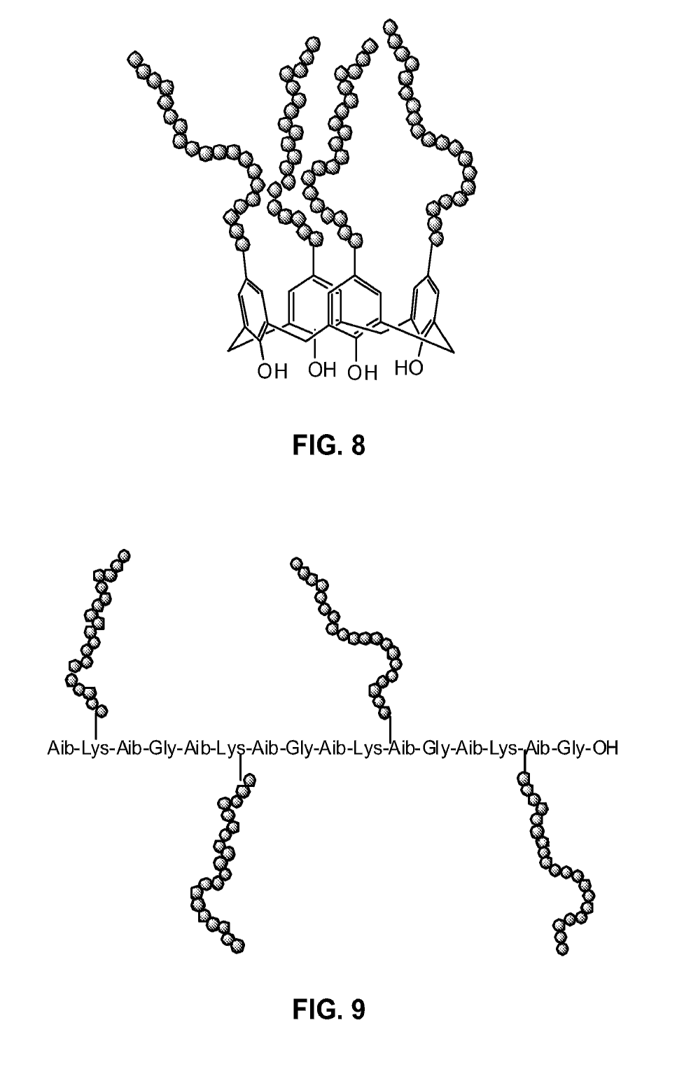

FIG. 9 illustrates an exemplary higher-order synbody wherein the linker is a polypeptide having a plurality of potentially reactive amino functions on the side chains. In FIG. 9, the residues bearing side-chain amino functions are lysines. In some embodiments, lower or even higher order homologs of lysine are employed (e.g., diaminopropionic acid (Dpr), diaminobutyric acid (Dub), ornithine (Orn), lysine, or homolysine). In some embodiments, unnatural (i.e., unphysiological) amino acids are incorporated into the linker. For example, the compound shown in FIG. 9 incorporates aminoisobutyric acid (Aib), which is known in the art to facilitate formation of alpha-helical secondary structure.

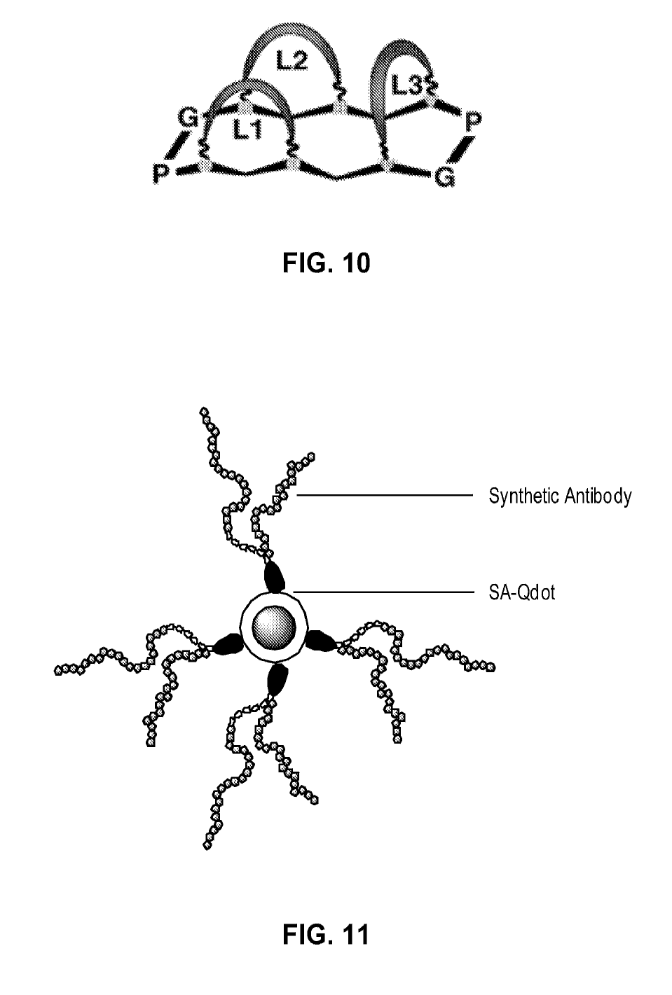

Another class of linker is solid particles. For example, nano-scale materials can provide versatile platform for construction of in vivo optical imaging probes. Semiconductor Q-dots have physical and optical properties that make them useful tool for imaging proteins in cells. Synbodies with a biotin adjunct molecule can be conjugated to such moieties for use as multivalent imaging agents (see FIG. 11). Synbodies with a biotin molecule can also be conjugated to streptavidin, which can be labeled (e.g., Alexa 555).

E. Adjunct Moieties

As well as two compounds that in combination effect binding to a target and a linker holding the compounds together, a synbody can include various adjunct moieties. The adjunct moieties are usually attached to the linker. An exemplary adjunct moiety is a biotin molecule attached via a lysine residue to another lysine residue to which first and second peptide are attached via the alpha and epsilon nitrogen atoms. The adjunct moiety can be a label, such as horse radish peroxidase, a moiety permitting immobilization such as biotin or a poly-his tag or FLAG® tag, a therapeutic moiety or a half-life extender, among others. Examples of half-life extenders including polyethylene glycol, phosphorylcholine, immunoglobulin constant region, and other blood proteins, such as serum albumin. Some examples of therapeutic moieties include antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, camptothecins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, the like. Individual cytotoxic or immunomodulatory agents include, for example, an androgen, anthramycin (AMC), asparaginase, 5-azacytidine, azathioprine, bleomycin, busulfan, buthionine sulfoximine, calicheamicin, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine and vinorelbine.

F. Screening of Variants

Selection of screening of amino acid variants has been described above. Variants including modifications of conjugation chemistry, linker, spacers or adjunct moieties can be screened for retention of binding to AKT1. In some methods, either the AKT1 or the synbody is immobilized. Optionally binding of a variant is tested in competition with the lead AKT1 synbody discussed above. In one format, AKT1 is immobilized to a support and synbody variants are tested for binding by surface plasmon resonance.

In another approach, peptides and/or variations in conjugation or linkage can be self-selected for desired binding in the presence of a target. The rationale idea behind this is approach is to allow peptides having complementary functionality to stitch to one another in a pair-wise fashion if two peptides are brought in close proximities by binding or cross linking to a protein target. Synbodies formed by this self-selection can be subject to further modification in amino acid sequence, conjugation chemistry or linkage.

G. Functional Properties

The invention provides synbodies representing any combination of the mutations, conjugation chemistries, linkers/scaffolds and adjunct moieties described above. The synbodies preferably bind to human AKT1 with an affinity of at least 107, 108 or 109 M-1. Some synbodies bind human AKT1 detectably more strongly than human AKT2 and human AKT3. Some synbodies have an affinity for human AKT1 at least ten times the affinity for human AKT or human AKT3. Some synbodies bind to human AKT1 such as to inhibit activation of human AKT1. Some synbodies bind to human AKT1 such as to inhibit it ability to phosphorylate other proteins.

III. Methods of Manufacture

In general, synbodies comprising affinity elements and linkers that can be synthesized by standard solid phase synthesis techniques can be synthesized either by addition of amino acids or other monomers in a stepwise fashion, or by joining preassembled affinity elements and linkers or other presynthesized subunits. Techniques for stepwise synthesis of peptides and other heteropolymers are described by e.g., Atherton E, Sheppard R C: Solid Phase peptide synthesis: a practical approach. Oxford, England: IRL Press; 1989, and Stewart J M, Young J D: Solid Phase Peptide Synthesis, 2d Ed. Rockford: Pierce Chemical Company; 1984, which are incorporated herein by reference. Examples of conjugation chemistries have been discussed above and in WO08/048970 and PCT/US2009/041570. The use of “click” chemistry to perform conjugations between biopolymers and other heteropolymers is also described in Kolb et al., Angewandte Chemie—International Edition 2001, 40(11):2004 and Evans, Australian Journal of Chemistry 2007, 60(6):384-395, which are incorporated herein by reference.

IV. Detection Methods

Synbodies binding to AKT1 are useful for detecting AKT1 as research reagents or diagnostics. Typically a synbody is contacted with a sample known or suspected to AKT1 and binding of the synbody to the sample is compared with a control. Binding can be assessed for example from a signal present on the synbody, the sample or a secondary labeling reagent The control is usually a negative control sample in which AKT1 is known to be absent. Stronger binding of the synbody to the sample relative to the negative control provides an indication that AKT1 is present. Alternatively or additionally, a positive control can be used in which a known amount of AKT1 is present. The relative binding of the AKT1 to the sample compared with the positive control provides an indication of presence or absence of AKT1 in the sample and if present, the amount.

In some methods, the sample is from a patient known or suspected to be suffering from cancer or to be at enhanced risk relative to the general population of developing cancer. The sample can be from a body fluid, such as blood (including plasma), CSF, urine, or milk, or a tumor, such as a breast tumor, ovarian tumor, pancreatic tumor or prostate tumor. Presence of a detectable level of AKT1 and particularly an elevated level of AKT1 relative to a noncancerous tissue matched sample from the same patient provides an indication of cancer, and an indication that the cancer is amenable to treatment with a synbody or other agent targeted against AKT1.

A variety of formats can be used for the assay, many analogous to formats used in immunological assays. These formats include immunoprecipitation or Western blotting with the synbody used in place of an antibody. Another format uses an immobilized or immobilizable synbody. In such a format, a signal indicative of binding can be provided by labeling the sample or by using a secondary detection reagent. The secondary detection reagent can be an antibody or another synbody binding to a different epitope on AKT1. Such a format is effectively a sandwich assay in which AKT1 is sandwiched between two synbodies or one synbody and an antibody. A sandwich assay can also be performed with an immobilized antibody and a synbody in solution as the detection reagent.

V. Treatment Methods and Compositions

Synbodies of the invention can be used to inhibit growth of cancers both in vitro and in patients. As discussed above AKT1 expression is elevated in several types of cancer including breast, ovarian, pancreatic and prostate. Although understanding of mechanism is not required for practice of invention, it is believed that synbodies can inhibit AKT1 by several different mechanisms including inhibition of its activation by other molecules, inhibition of its own activity in phosphorylating other proteins and by using synbodies as a means to target another therapeutic molecule, such as discussed above to a cancer.

Synbodies are most useful for treating cancers in which AKT1 expression can be detected at either the mRNA or protein level, and particularly cancers in which AKT1 expression is elevated relative to tissue-matched noncancerous tissues in the same patient. In some methods, expression of AKT1 in a cancer is checked, optionally in comparison with expression of a tissue matched noncancerous sample from the same patient. However, checking the expression level is not required.

Alternatively or additionally, the level of AKT kinase activity in a cancer cell can be quantified using an in vitro kinase assay. A variety of AKT kinase assay kits are commercially available, for example, from BioSource International, BioVision, Calbiochem, Cell Signaling Technology, Molecular Devices, Upstate Biotechnology, or Stressgen Biologicals. Detectable activity of AKT1 kinase and particularly elevated activity relative to a tissue matched noncancerous control sample provide an indication that cancer is amenable to treatment with synbodies of the invention.

Increased copy number of the AKT1 gene in a cancer cell can provide a further indication that a caner is amenable to treatment. Increased copy number can be detected using for example, Southern blotting, quantitative PCR, fluorescence in situ hybridization of metaphase chromosome spreads, and other cytogenetic techniques.

Types of cancer potentially amenable to treatment include, ovarian cancer, breast cancer, lung cancer (small cell or non-small cell), colon cancer, prostate cancer, pancreatic cancer, renal cancer, gastric cancer, particularly adenocarcinoma, liver cancer, head-and-neck tumors, melanoma, sarcomas, and brain tumors (e.g., glioblastomas), of children or adults. Treatment can also be administered to patients having leukemias, e.g., chronic myelogenous or lymphomas.

Synbodies, typically in a pharmaceutical formulation can be administered to a patient by any suitable route, especially parentally by intravenous infusion or bolus injection, intramuscularly or subcutaneously. The synbody can also be injected directly into the site of disease (e.g., a tumor), or encapsulated into carrying agents such as liposomes. The dose given is sufficient to alleviate the condition being treated (“therapeutically effective dose”) and can be, for example, 0.1-10 mg/kg body weight. fixed unit dose may also be given, for example, 50-1000 mg, or the dose can be based on the patient's surface area, e.g., 100 mg/m2. Usually between 1 and 8 doses are administered to treat cancer, but more doses can be given. The synbody can be administered daily, biweekly, weekly, every other week, monthly or at some other interval, depending, e.g. on the half-life of the synbody for 1 week, 2 weeks, 4 weeks, 8 weeks, 3-6 months or longer. Repeated courses of treatment are also possible, as is chronic administration. A regime of a dosage and intervals of administration that alleviates or at least partially arrests the symptoms of the disease (biochemical, histologic and/or clinical), including its complications and intermediate pathological phenotypes in development of the disease is referred to as a therapeutically effective regime.

Synbodies can also be used in prophylaxis of a patient at risk of cancer. Such patients include those having genetic susceptibility to cancer, patients who have undergone exposure to carcinogenic agents, such as radiation or toxins, and patients who have undergone previous treatment for cancer and are at risk of recurrence. A prophylactic dosage is an amount sufficient to eliminate or reduce the risk, lessen the severity, or delay the outset of the disease, including biochemical, histologic and/or clinical symptoms of the disease, its complications and intermediate pathological phenotypes presenting during development of the disease. Administration of a pharmaceutical composition in an amount and at intervals effective to effect one or more of these objects is referred to as a prophylactically effective regime.

Treatment with a synbody can be combined with conventional treatments, for example Taxol (paclitaxel) or its derivatives, platinum compounds such as carboplatin or cisplatin, anthrocyclines such as doxorubicin, alkylating agents such as cyclophosphamide, anti-metabolites such as 5-fluorouracil, or etoposide. A synbody can be administered in combination with two, three or more of these agents in a standard chemotherapeutic regimen, for example, taxol and carboplatin, e.g. for breast and ovarian cancer. Other agents with which the synbody can be administered include biologics such as monoclonal antibodies, including Herceptin™ against the HER2 antigen, Avastin™ against VEGF, or antibodies to the EGF receptor, as well as small molecule anti-angiogenic or EGF receptor antagonist drugs. In addition, the synbody can be used together with radiation therapy or surgery.

Treatment including the synbody may increase the median progression-free survival or overall survival time of patients with a cancer by at least 30% or 40% but preferably 50%, 60% to 70% or even 100% or longer, compared to an otherwise comparable regime but without the synbody. In addition or alternatively, treatment including the synbody may increase the complete response rate, partial response rate, or objective response rate (complete+partial) of patients with a cancer (e.g., ovarian, breast, pancreas especially when relapsed or refractory) by at least 30% or 40% but preferably 50%, 60% to 70% or even 100% compared to the same regime without the synbody. Optionally, treatment can inhibit tumor invasion, or metastasis.

Typically, in a clinical trial (e.g., a phase II, phase II/III or phase III trial), the increases in median progression-free survival and/or response rate of the patients treated with chemotherapy plus a synbody relative to the control group of patients receiving chemotherapy alone (or plus placebo) is statistically significant, for example at the p=0.05 or 0.01 or even 0.001 level. The complete and partial response rates are determined by objective criteria commonly used in clinical trials for cancer, e.g., as listed or accepted by the National Cancer Institute and/or Food and Drug Administration.

Synbodies can be administered in the form of a pharmaceutical composition. Pharmaceutical compositions are typically manufactured under GMP condition. Pharmaceutical compositions can be provided in unit dosage form (i.e., the dosage for a single administration). Pharmaceutical compositions can be formulated using one or more physiologically acceptable carriers, diluents, excipients or auxiliaries. The formulation dependent on the route of administration chosen.

Administration can be parenteral, intravenous, oral, subcutaneous, intra-arterial, intracranial, intrathecal, intraperitoneal, topical, intranasal or intramuscular. Pharmaceutical compositions for parenteral administration are preferably sterile and substantially isotonic. For injection, synbodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively synbodies can be in powder form for constitution with a suitable vehicle, e.g., sterile pyrogen-free water, before use.

### EXAMPLES

### Example 1

### Properties of Lead Synbody

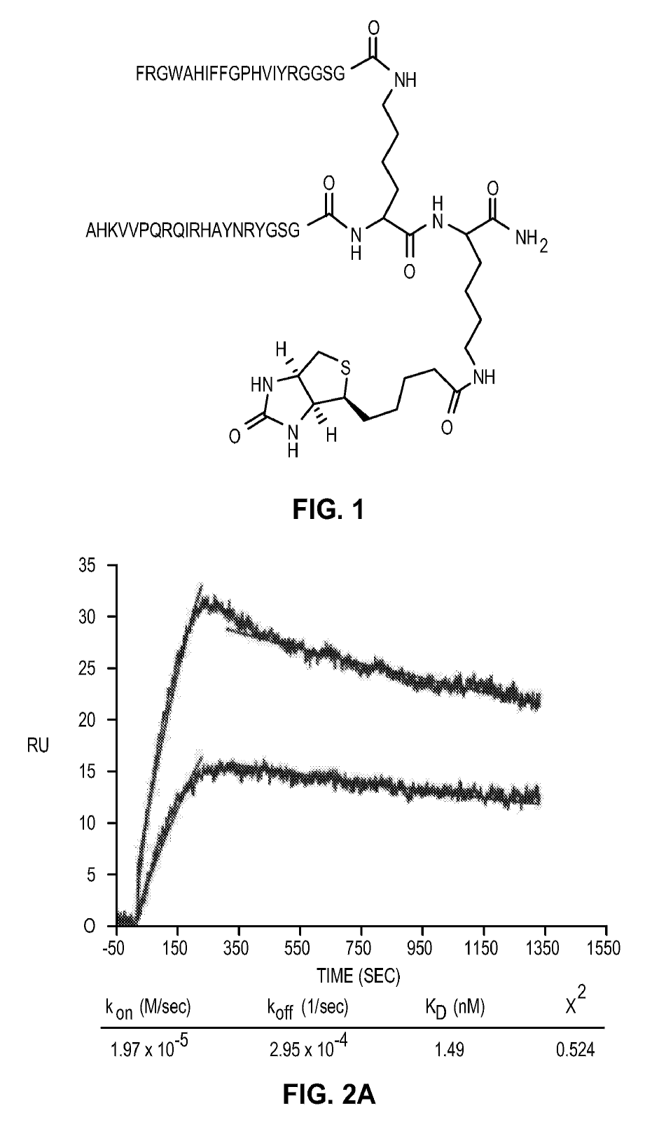

FIG. 1 shows an exemplary synbody in which peptides Akt26 (AHKVVPQRQIRHAYNRYGSG) (SEQ ID NO: 2) and Akt23 (FRGWAHIFFGPHVIYRGGSG) (SEQ ID NO: 3) are linked via amide bonds to the alpha and epsilon carbon atoms of a lysine. The lysine is in turn linked by an amide bond to a second lysine molecule, which is in turn linked to a biotin molecule. The two 20 amino acid specific peptides are attached via standard Fmoc divergent (modified) solid phase peptide synthesis using orthogonal protecting groups on branched lysine. Two orthogonal groups are introduced using Fmoc-Lys(ivDde)-OH at the very C-terminus. The stepwise assembly of the peptide sequences Akt26 and Akt23 is accomplished at Nα and Nε-positions respectively.

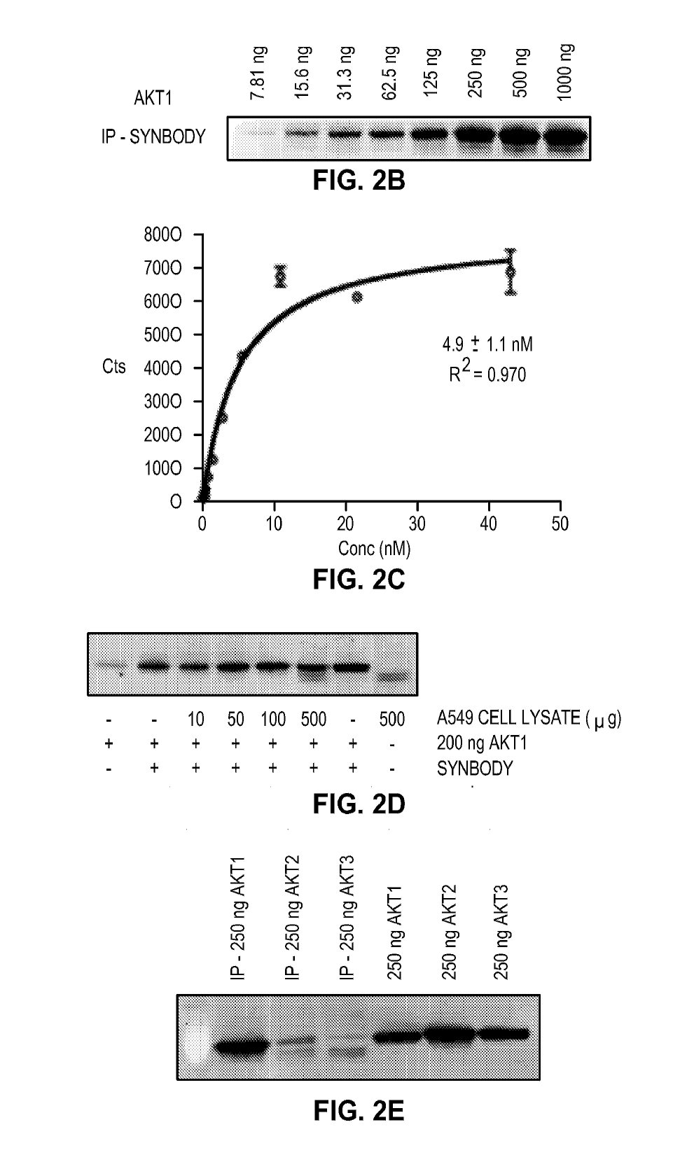

The synbody was tested for binding to AKT1 and in some experiments, AKT2 and AKT3. FIG. 2A shows SPR sensorgrams from 12.5 and 6.25 nM synbody injected over AKT1 surface. A kinetic fit of the data using a 1:1 binding model is shown in red and yields a K_{D} of 1.49 nM with a χ² value of 0.524. FIG. 2B shows a Western Blot of immunoprecipitation (IP) of AKT1 using the synbody. FIG. 2C shows a plot of S³⁵-labeled AKT1 pulled-down with synbody immobilized via its biotin to a magnetic bead. The data were fit to a 1:1 binding model and yielded a K_{D} of 4.9±1.1 nM. FIG. 2D) shows a Western Blot of 200 ng of recombinant AKT1 pulled down in the presence of increasing concentrations of A549 cell lysate. The outside lane demonstrates the precipitation of native AKT1 from 500 mg of cell lysate. FIG. 2E shows immunoprecipitate of purified 250 ng of AKT1, AKT2, and AKT3 from 500 mg of cell lysate. The synbody shows little cross-reactivity with AKT2 and AKT3.

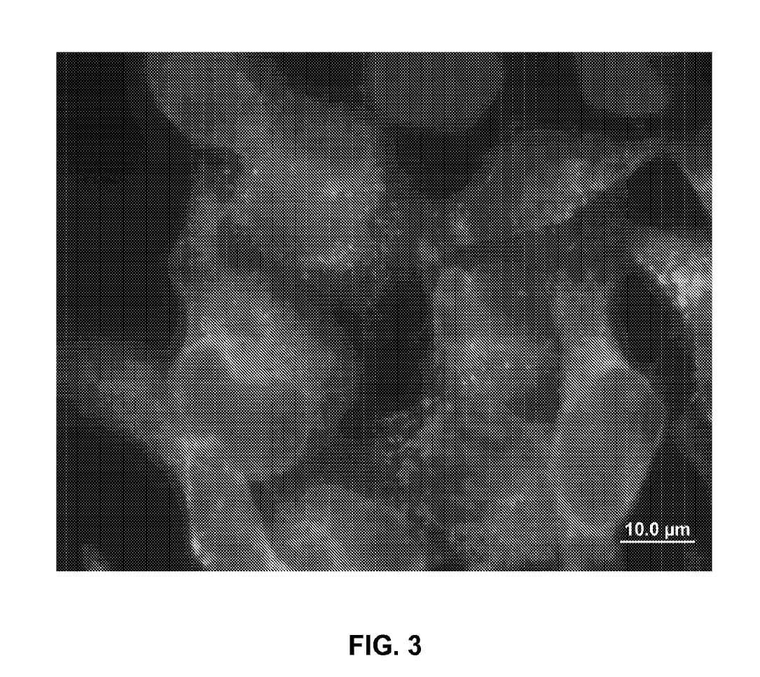

The synbody was also tested for ability to detect Akt1 by Immunofluorescence microscopy: TE671 cells were grown on 8-well glass slide. Cells were then washed 3 times in PBS and fixed by incubating at room temperature (RT) in 1% formaldehyde solution. After fixation, cells were treated with PBST containing 0.1% triton X-100 and blocked in 1% BSA for 1 hour at RT. Cells were stained with 50 nM 26-23-(PEG2) Alexafluor 488 at RT for 1 hr. Cells were washed five times with PBST, stained with DAPI-antifade. Slides were stored overnight in dark and then imaged on epifluorescence microscope. FIG. 3 shows the immunofluorescence image of TE671 cells using the synbody.

### Example 2

### Variants of Lead Synbody

The following synbodies have the same peptides as the lead synbody and are synthesized off of a lysine core but include the addition of different functional groups synthesized between the C-terminus of the peptide and the amine groups of the lysine scaffold (Table 2). Synbodies were conjugated with either a C-terminal cysteine protected against disulfide formation using a sulfo-tert-butyl group, or with different biotin-conjugated lysines where the spacer between the lysine and the backbone varied. For example, in synbody 5 the biotin was attached to the amino group of the side chain, synbody 6 used a caproic acid spacer, while synbody 7 used an eleven-unit polyethylene glycol spacer.

TABLE 2
Synbodies constructed from a lysine scaffold.

| K_{D} | | | |
|---|---|---|---|
| (nM) | Synbody Name | Amino Acid Sequence | |
| 4 | Akt26-23KC | AHKVVPQRQIRHAYNRYGSGKCFRGWAHIFFGPHVIYRGGSG | (SEQ ID NO: 14) |
| n.d. | Akt23-26KC | FRGWAHIFFGPHVIYRGGSGKCAHKVVPQRQIRHAYNRYGSG | (SEQ ID NO: 15) |
| n.d. | Akt23-23KC | FRGWAHIFFGPHVIYRGGSGKCFRGWAHIFFGPHVIYRGGSG | (SEQ ID NO: 16) |
| n.d. | Akt26-26KC | AHKVVPQRQIRHAYNRYGSGKCAHKVVPQRQIRHAYNRYGSG | (SEQ ID NO: 17) |
| 4 | Akt26- | AHKVVPQRQIRHAYNRYGSGKKFRGWAHIFFGPHVIYRGGSG | (SEQ ID NO: 18) |
| | 23KKbiotin | | |
| n.d. | Akt26- | AHKVVPQRQIRHAYNRYGSGKKFRGWAHIFFGPHVIYRGGSG | (SEQ ID NO: 19) |
| | 23KKCapBiotin | | |
| n.d. | Akt26- | AHKVVPQRQIRHAYNRYGSGKKFRGWAHIFFGPHVIYRGGSG | (SEQ ID NO: 20) |
| | 23KE(PEGBiotin) | | |

Synbodies Extended with Octanoic and Hexanoic Acid

| | | | |
|---|---|---|---|
| 6 | Akt26(Oct)-23KC | AHKVVPQRQIRHAYNRYGSG(oct)KCFRGWAHIFFGPHVIYRGGSG | (SEQ ID NO: 21) |
| n.b. | Akt26-23(Oct)KC | AHKVVPQRQIRHAYNRYGSGKCFRGWAHIFFGPHVIYRGGSG(oct) | (SEQ ID NO: 22) |
| n.b. | Akt26-(Oct)- | AHKVVPQRQIRHAYNRYGSG(oct)KCFRGWAHIFFGPHVIYRGGSG(oct) | (SEQ ID NO: 23) |
| | 23(Oct)KC | | |
| 3 | Akt26-23(hex)KC | AHKVVPQRQIRHAYNRYGSGKCFRGWAHIFFGPHVIYRGGSG(hex) | (SEQ ID NO: 24) |
| 100 | Akt26(Oct)- | AHKVVPQRQIRHAYNRYGSG(oct)KCFRGWAHIFFGPHVIYRGGSG(hex) | (SEQ ID NO: 25) |
| | 23(hex)KC | | |

Synbodies Extended with (poly-ethylene glycol)ₙ

| | | | |
|---|---|---|---|
| n.d. | Akt 26(PEG)n- | AHKVVPQRQIRHAYNRYGSG(PEG)ₙKCFRGWAHIFFGPHVIYRGGSG | (SEQ ID NO: 26) |
| | 23KC (n = 1-8) | | |
| n.d. | Akt26- | AHKVVPQRQIRHAYNRYGSGKCFRGWAHIFFGPHVIYRGGSG(PEG)ₙ | (SEQ ID NO: 27) |
| | 23(PEG)nKC | | |
| | (n = 1-8) | | |
| n.d. | Akt26(PEG)n- | AHKVVPQRQIRHAYNRYGSG(PEG)KCFRGWAHIFFGPHVIYRGGSG(PEG)ₙ | (SEQ ID NO: 28) |
| | 23(PEG)KC | | |
| | (n = 1-8) | | |

Synbodies Extended with (Pro-Gly-Pro)ₙ

| | | | |
|---|---|---|---|
| n.d. | Akt26-[(PGP)n]- | AHKVVPQRQIRHAYNRYGSG(PGP)nKCFRGWAHIFFGPHVIYRGGSG | (SEQ ID NO: 29) |
| | 23KC (n = 1-8) | | |
| n.d. | Akt26- | AHKVVPQRQIRHAYNRYGSGKCFRGWAHIFFGPHVIYRGGSG(PGP)n | (SEQ ID NO: 30) |
| | 23(PGP)nKC | | |
| | (n = 1-8) | | |

Synbodies Extended with (Pro-Pro-Pro)ₙ

| | | | |
|---|---|---|---|
| n.d. | Akt26-[(PPP)n]- | AHKVVPQRQIRHAYNRYGSG(PPP)nKCFRGWAHIFFGPHVIYRGGSG | (SEQ ID NO: 31) |
| | 23KC (n = 1-8) | | |
| n.d. | Akt26- | AHKVVPQRQIRHAYNRYGSGKCFRGWAHIFFGPHVIYRGGSG(PPP)n | (SEQ ID NO: 32) |
| | 23(PPP)nKC | | |
| | (n = 1-8) | | |

Synbodies Extended with β-Amino Hexanoic Acid

| | | | |
|---|---|---|---|
| n.d. | Akt26(βAha)- | AHKVVPQRQIRHAYNRYGSG(βAha)KCFRGWAHIFFGPHVIYRGGSG | (SEQ ID NO: 33) |
| | 23KC | | |
| n.d. | Akt26- | AHKVVPQRQIRHAYNRYGSGKCFRGWAHIFFGPHVIYRGGSG(βAha) | (SEQ ID NO: 34) |
| | 23(βAha)KC | | |
| n.d. | Akt26(βAha)- | AHKVVPQRQIRHAYNRYGSG(βAha)KCFRGWAHIFFGPHVIIIIGGSG(βAha) | (SEQ ID NO: 35) |
| | 23(βAha)KC | | |

n.b. = no binding
n.d. = not determined

The following synbodies were constructed from the same peptides as the lead synbody except were constructed using the triazole coupling chemistry and varying scaffolds (Table 2). The following scaffolds were used: repeat units of (Pro-Gly-Pro)ₙ (n=1-6); repeat units of (Pro-Pro-Pro)ₙ (n=1-7); and repeat units of poly-ethylene glycol (PEG)ₙ (n=1-7).

TABLE 3
Synbodies constructed by Triazole Coupling Chemistry

| KD | | | |
|---|---|---|---|
| (nM) | Synbody Name | Synbodies Constructed by Triazole Coupling | |
| n.d. | Akt26-23(PGP)ₙK(Biotin) | AHKVVPQRQIRHAYNRYGSG(PGP)ₙKKFRGWAHIFFGPHVIYRGKSG | (SEQ ID NO: 36) |
| | (n = 1-6) | | |
| n.d. | Akt 26-23(PGP)ₙC | AHKVVPQRQIRHAYNRYGSG(PGP)ₙKCFRGWAHIFFGPHVIYRGKSG | (SEQ ID NO: 37) |
| | (n = 1-6) | | |
| n.d. | Akt 26-23(PPP)ₙKGA | AHKVVPQRQIRHAYNRYGSG(PPP)ₙKGAFRGWAHIFFGPHVIYRGKSG | (SEQ ID NO: 38) |
| | (n = 1-5) | | |
| n.d. | Akt 26-23(PPP)ₙKC | AHKVVPQRQIRHAYNRYGSG(PPP)ₙKKFRGWAHIFFGPHVIYRGKSG | (SEQ ID NO: 39) |
| | (n = 1-7) | | |
| n.d. | m Akt 26-23(PGP)₄KC | AHKVVYQRQIRFAYNRYGSG(PGP)₄KCFRGWAHIFFGPHVIYRGKSG | (SEQ ID NO: 40) |
| n.d. | m Akt 26-23(PPP)₂KC | AHKVVYQRQIRFAYNRYGSG(PPP)₂KCFRGWAHIFFGPHVIYRGKSG | (SEQ ID NO: 41) |
| n.d. | m Akt 26-23(PPP)₂KC | AHKVVYQRQIRFAYNRYGSG(PPP)₂KCFRGWAHIFFGPHVIYRGKSG | (SEQ ID NO: 42) |
| n.d. | Akt 26-23(PEG)ₙKC | AHKVVPQRQIRHAYNRYGSG(PEG)₁KCFRGWAHIFFGPHVIYRGKSG | (SEQ ID NO: 43) |
| | (n = 1-7) | | |

The following mutant forms of the lead peptide have been made in which either one or more amino acids are substituted or the orientation of the peptides on the linker is reversed (Table 4).

TABLE 4
Synbodies constructed from mutant peptides on lysine scaffold

| KD | | | |
|---|---|---|---|
| (nM) | Synbody Name | Amino Acid Sequence | |
| 15 | m1-Akt26-23 KC | AHKVVPQRQMRHAYSRYGSGKCFRGWAHIFFGPHVIYRGGSG | (SEQ ID NO: 44) |
| 5 | m2-Akt26-23 KC | AHKVVYQRQIRFAYNRYGSGKCFRGWAHIFFGPHVIYRGGSG | (SEQ ID NO: 45) |
| n.d. | Akt23-m2-26 KC | FRGWAHIFFGPHVIYRGGSGKCAHKVVYQRQIRFAYNRYGSG | (SEQ ID NO: 46) |
| n.b. | m3-Akt26-23 KC | AWKVVPQRQIRHAYNRYGSGKCFRGWAHIFFGPHVIYRGGSG | (SEQ ID NO: 47) |
| 5 | m4-Akt26-23 KC | AHKVVPQRWIRHAYNRYGSGKCFRGWAHIFFGPHVIYRGGSG | (SEQ ID NO: 48) |
| 8 | m5-Akt26-23 KC | AWKVVPQRWIRHAYNRYGSGKCFRGWAHIFFGPHVIYRGGSG | (SEQ ID NO: 49) |
| ~10 | m6-Akt26-23 KC | AWKVVPQRWIRYAYNRYGSGKCFRGWAHIFFGPHVIYRGGSG | (SEQ ID NO: 50) |

Although the invention has been described with reference to the presently preferred embodiments, it should be understood that various modifications can be made without departing from the invention. Unless otherwise apparent from the context any step, element, embodiment, feature or aspect of the invention can be used with any other. All publications (including GenBank or Swiss-Prot Accession numbers and the like), patents and patent applications cited are herein incorporated by reference in their entirety for all purposes to the same extent as if each individual publication, patent and patent application was specifically and individually indicated to be incorporated by reference in its entirety for all purposes. If more than one version of a sequence is associated with a deposit number at different times, the version associated with the deposit number at the time of filing the application is meant.

What is claimed is:

1. An agent comprising a first peptide having an amino acid sequence comprising AX₁KVVX₂QRX₃X₄RX₅AYX₆RYGSG (SEQ ID NO: 1), wherein X₁ is H or W, X₂ is P or Y, X₃ is Q or W, X₄ is I or M, X₅ is H, Y or F, and X₆ is N or S, and a second peptide having an amino acid sequence comprising FRGWAHIFFGPHVIYRGGSG (SEQ ID NO: 3), and a linker joining the first and second peptides, provided that up to four amino acids can be substituted for, inserted or deleted in the first and/or second peptides at positions not indicated by X, and provided that the first peptide does not have an amino acid sequence consisting of AHKVVPQRQIRHAYNRYGSG (SEQ ID NO: 2) or the first and second peptides are not linked via amide bonds to alpha and epsilon amino groups of a lysine linker.

2. The agent of claim 1 that has an affinity for human AKT1 of at least 10⁸ M⁻¹.

3. The agent of claim 1 that has an affinity for human AKT1 of at least 10⁹ M⁻¹.

4. The agent of claim 1 wherein the first and second peptides are linked in a MAP format.

5. The agent of claim 1, wherein the linker is an amino acid, peptide, polymer, a cyclic compound, or a particle.

6. The agent of claim 1, wherein the linker is a lysine, dilysine, lysine-cysteine, PGP, PEG, a sequential oigo-peptide carrier, a templated assembled scaffold, amino biphenyl carboxylic acid, a calyx(n)arene, triazacylophane, beta-cyclodextrine, a nanoparticle, a gold particle, or a quantum dot.

7. The agent of claim 1, comprising at least two molecules of the first and/or second peptide, wherein each peptide is linked to the linker.

8. The agent of claim 1, wherein the linker is polylysine.

9. The agent of claim 1, further comprising a label, immobilizing moiety, therapeutic molecule or half-life extender.

10. The agent of claim 9, wherein the label, immobilizing moiety, therapeutic molecule or half-life extender is attached to the linker.

11. The agent of claim 9, wherein the immobilizing moiety is biotin.

12. The agent of claim 9, wherein the therapeutic molecule is a cytotoxic molecule.

13. The agent of claim 9, wherein the half-life extender is selected from PEG, phosphorylcholine and an immunoglobulin constant region.

14. A method of detecting AKT1, comprising
contacting a sample suspected of containing AKT1 with an agent of claim 1, and measuring binding of the agent to the sample compared with a control lacking AKT1, an increase in binding relative to the control providing an indication of presence of AKT1.

15. The method of claim 14, wherein the sample is from a patient having or suspected of having a cancer or elevated risk of cancer.

16. The method of claim 14, further comprising contacting the sample with a second agent that binds a different epitope of AKT1, wherein either the first or second agent is immobilized and wherein the measuring step detects a sandwich formed between the first agent, AKT1 and the second agent.

17. A method of inhibiting growth of a cancer comprising, contacting the cancer with the agent of claim 1.

18. The method of claim 17, wherein the agent further comprises a therapeutic molecule linked to the linker.

19. The method of claim 17, wherein the cancer is present in a patient.

20. The method of claim 19, wherein the cancer is ovarian, breast or prostate cancer.

21. An isolated peptide having an amino acid sequence comprising or consisting of AHKVVPQRQMRHAYSRYGSG (SEQ ID NO: 57), AHKVVYQRQIRFAYNRYGSG (SEQ ID NO: 58), AWKVVPQRQIRHAYNRYGSG (SEQ ID NO: 59), AHKVVPQRWIRHAYNRYGSG (SEQ ID NO: 60), AWKVVPQRWIRHAYNRYGSG (SEQ ID NO: 61), AWKVVPQRWIRYAYNRYGSG (SEQ ID NO: 62), or CAHKVVYQRQIRFAYNRYGSG (SEQ ID NO: 63).


SEQUENCE LISTING (63 sequences)

SEQ ID NO: 1 - protein, 20 aa, Artificial Sequence
  Synthetic antibodies
AXKVVXQRXXRXAYXRYGSG

SEQ ID NO: 2 - protein, 20 aa, Artificial Sequence
  Synthetic antibodies
AHKVVPQRQIRHAYNRYGSG

SEQ ID NO: 3 - protein, 20 aa, Artificial Sequence
  Synthetic antibodies
FRGWAHIFFGPHVIYRGGSG

SEQ ID NO: 4 - protein, 480 aa, Homo sapiens [sequence omitted]

SEQ ID NO: 5 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 6 - protein, 479 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE

SEQ ID NO: 7 - protein, 42 aa, Artificial Sequence
  Synthetic antibodies
AHKVVPQRQMRHAYSRYGSGKCFRGWAHIFFGPHVIYRGGSG

SEQ ID NO: 8 - protein, 42 aa, Artificial Sequence
  Synthetic antibodies
AHKVVYQRQIRFAYNRYGSGKCFRGWAHIFFGPHVIYRGGSG

SEQ ID NO: 9 - protein, 42 aa, Artificial Sequence
  Synthetic antibodies
FRGWAHIFFGPHVIYRGGSGKCAHKVVYQRQIRFAYNRYGSG

SEQ ID NO: 10 - protein, 42 aa, Artificial Sequence
  Synthetic antibodies
AWKVVPQRQIRHAYNRYGSGKCFRGWAHIFFGPHVIYRGGSG

SEQ ID NO: 11 - protein, 42 aa, Artificial Sequence
  Synthetic antibodies
AHKVVPQRWIRHAYNRYGSGKCFRGWAHIFFGPHVIYRGGSG

SEQ ID NO: 12 - protein, 42 aa, Artificial Sequence; identical to SEQ ID NO: 11
  Synthetic antibodies

SEQ ID NO: 13 - protein, 42 aa, Artificial Sequence
  Synthetic antibodies
AWKVVPQRWIRYAYNRYGSGKCFRGWAHIFFGPHVIYRGGSG

SEQ ID NO: 14 - protein, 42 aa, Artificial Sequence
  Synthetic antibodies
AHKVVPQRQIRHAYNRYGSGKCFRGWAHIFFGPHVIYRGGSG

SEQ ID NO: 15 - protein, 42 aa, Artificial Sequence
  Synthetic antibodies
FRGWAHIFFGPHVIYRGGSGKCAHKVVPQRQIRHAYNRYGSG

SEQ ID NO: 16 - protein, 42 aa, Artificial Sequence
  Synthetic antibodies
FRGWAHIFFGPHVIYRGGSGKCFRGWAHIFFGPHVIYRGGSG

SEQ ID NO: 17 - protein, 42 aa, Artificial Sequence
  Synthetic antibodies
AHKVVPQRQIRHAYNRYGSGKCAHKVVPQRQIRHAYNRYGSG

SEQ ID NO: 18 - protein, 42 aa, Artificial Sequence
  Synthetic antibodies
AHKVVPQRQIRHAYNRYGSGKKFRGWAHIFFGPHVIYRGGSG

SEQ ID NO: 19 - protein, 42 aa, Artificial Sequence; identical to SEQ ID NO: 18
  Synthetic antibodies

SEQ ID NO: 20 - protein, 42 aa, Artificial Sequence; identical to SEQ ID NO: 18
  Synthetic antibodies

SEQ ID NO: 21 - protein, 42 aa, Artificial Sequence; identical to SEQ ID NO: 14
  Synthetic antibodies

SEQ ID NO: 22 - protein, 42 aa, Artificial Sequence; identical to SEQ ID NO: 14
  Synthetic antibodies

SEQ ID NO: 23 - protein, 42 aa, Artificial Sequence; identical to SEQ ID NO: 14
  Synthetic antibodies

SEQ ID NO: 24 - protein, 42 aa, Artificial Sequence; identical to SEQ ID NO: 14
  Synthetic antibodies

SEQ ID NO: 25 - protein, 42 aa, Artificial Sequence; identical to SEQ ID NO: 14
  Synthetic antibodies

SEQ ID NO: 26 - protein, 42 aa, Artificial Sequence; identical to SEQ ID NO: 14
  Synthetic antibodies

SEQ ID NO: 27 - protein, 42 aa, Artificial Sequence; identical to SEQ ID NO: 14
  Synthetic antibodies

SEQ ID NO: 28 - protein, 42 aa, Artificial Sequence; identical to SEQ ID NO: 14
  Synthetic antibodies

SEQ ID NO: 29 - protein, 66 aa, Artificial Sequence
  Synthetic antibodies
AHKVVPQRQIRHAYNRYGSGPGPPGPPGPPGPPGPPGPPGPPGPKCFRGWAHIFFGPHVIYRGGSG

SEQ ID NO: 30 - protein, 66 aa, Artificial Sequence
  Synthetic antibodies
AHKVVPQRQIRHAYNRYGSGKCFRGWAHIFFGPHVIYRGGSGPGPPGPPGPPGPPGPPGPPGPPGP

SEQ ID NO: 31 - protein, 66 aa, Artificial Sequence
  Synthetic antibodies
AHKVVPQRQIRHAYNRYGSGPPPPPPPPPPPPPPPPPPPPPPPPKCFRGWAHIFFGPHVIYRGGSG

SEQ ID NO: 32 - protein, 66 aa, Artificial Sequence
  Synthetic antibodies
AHKVVPQRQIRHAYNRYGSGKCFRGWAHIFFGPHVIYRGGSGPPPPPPPPPPPPPPPPPPPPPPPP

SEQ ID NO: 33 - protein, 42 aa, Artificial Sequence; identical to SEQ ID NO: 14
  Synthetic antibodies

SEQ ID NO: 34 - protein, 42 aa, Artificial Sequence; identical to SEQ ID NO: 14
  Synthetic antibodies

SEQ ID NO: 35 - protein, 42 aa, Artificial Sequence; identical to SEQ ID NO: 14
  Synthetic antibodies

SEQ ID NO: 36 - protein, 60 aa, Artificial Sequence
  Synthetic peptide
AHKVVPQRQIRHAYNRYGSGPGPPGPPGPPGPPGPPGPKKFRGWAHIFFGPHVIYRGKSG

SEQ ID NO: 37 - protein, 60 aa, Artificial Sequence
  Synthetic peptide
AHKVVPQRQIRHAYNRYGSGPGPPGPPGPPGPPGPPGPKCFRGWAHIFFGPHVIYRGKSG

SEQ ID NO: 38 - protein, 58 aa, Artificial Sequence
  Synthetic peptide
AHKVVPQRQIRHAYNRYGSGPPPPPPPPPPPPPPPKGAFRGWAHIFFGPHVIYRGKSG

SEQ ID NO: 39 - protein, 63 aa, Artificial Sequence
  Synthetic peptide
AHKVVPQRQIRHAYNRYGSGPPPPPPPPPPPPPPPPPPPPPKKFRGWAHIFFGPHVIYRGKSG

SEQ ID NO: 40 - protein, 54 aa, Artificial Sequence
  Synthetic peptide
AHKVVYQRQIRFAYNRYGSGPGPPGPPGPPGPKCFRGWAHIFFGPHVIYRGKSG

SEQ ID NO: 41 - protein, 48 aa, Artificial Sequence
  Synthetic peptide
AHKVVYQRQIRFAYNRYGSGPPPPPPKCFRGWAHIFFGPHVIYRGKSG

SEQ ID NO: 42 - protein, 48 aa, Artificial Sequence; identical to SEQ ID NO: 41
  Synthetic peptide

SEQ ID NO: 43 - protein, 42 aa, Artificial Sequence
  Synthetic antibodies
AHKVVPQRQIRHAYNRYGSGKCFRGWAHIFFGPHVIYRGKSG

SEQ ID NO: 44 - protein, 42 aa, Artificial Sequence; identical to SEQ ID NO: 7
  Synthetic antibodies

SEQ ID NO: 45 - protein, 42 aa, Artificial Sequence; identical to SEQ ID NO: 8
  Synthetic antibodies

SEQ ID NO: 46 - protein, 42 aa, Artificial Sequence; identical to SEQ ID NO: 9
  Synthetic antibodies

SEQ ID NO: 47 - protein, 42 aa, Artificial Sequence; identical to SEQ ID NO: 10
  Synthetic antibodies

SEQ ID NO: 48 - protein, 42 aa, Artificial Sequence; identical to SEQ ID NO: 11
  Synthetic antibodies

SEQ ID NO: 49 - protein, 42 aa, Artificial Sequence
  Synthetic antibodies
AWKVVPQRWIRHAYNRYGSGKCFRGWAHIFFGPHVIYRGGSG

SEQ ID NO: 50 - protein, 42 aa, Artificial Sequence; identical to SEQ ID NO: 13
  Synthetic antibodies

SEQ ID NO: 51 - protein, 23 aa, Artificial Sequence
  Synthetic peptide
FRGWAHIFFGPHVIYRGGSGKSG

SEQ ID NO: 52 - protein, 20 aa, Artificial Sequence
  Synthetic peptide
FRGWAHIFFGPHVIYRGKSG

SEQ ID NO: 53 - protein, 23 aa, Artificial Sequence
  Synthetic peptide
AHKVVPQRQIRHAYNRYGSGKSG

SEQ ID NO: 54 - protein, 23 aa, Artificial Sequence
  Synthetic peptide
FRGWAHIFFGPHVIYRGGSGYSG

SEQ ID NO: 55 - protein, 32 aa, Artificial Sequence
  Synthetic peptide
AHKVVPQRQIRHAYNRYGSGPGPPGPPGPPGP

SEQ ID NO: 56 - protein, 20 aa, Artificial Sequence
  Synthetic peptide
FRGWAHIFFGPHVIYRGKSC

SEQ ID NO: 57 - protein, 20 aa, Artificial Sequence
  Synthetic peptide
AHKVVPQRQMRHAYSRYGSG

SEQ ID NO: 58 - protein, 20 aa, Artificial Sequence
  Synthetic peptide
AHKVVYQRQIRFAYNRYGSG

SEQ ID NO: 59 - protein, 20 aa, Artificial Sequence
  Synthetic peptide
AWKVVPQRQIRHAYNRYGSG

SEQ ID NO: 60 - protein, 20 aa, Artificial Sequence
  Synthetic peptide
AHKVVPQRWIRHAYNRYGSG

SEQ ID NO: 61 - protein, 20 aa, Artificial Sequence
  Synthetic peptide
AWKVVPQRWIRHAYNRYGSG

SEQ ID NO: 62 - protein, 20 aa, Artificial Sequence
  Synthetic peptide
AWKVVPQRWIRYAYNRYGSG

SEQ ID NO: 63 - protein, 21 aa, Artificial Sequence
  Synthetic peptide
CAHKVVYQRQIRFAYNRYGSG